(19) United States
(12) Patent Application Publication
(10) Pub. No.: US 2004/0242496 A1
(43) Pub. Date: Dec. 2, 2004

(54) Phosphate suspensions

(76) Inventor: John Hawkins, South Staffs (GB)

Correspondence Address: Christopher J Whewell, Legal Department, Huntsman, P O Box 15730, Austin, TX, 78761, US

(21) Appl. No.: 10/483,926

(22) PCT Filed: Jul. 12, 2002
(86) PCT No.: PCT/EP02/07786

(30) Foreign Application Priority Data
Jul. 20, 2001 (GB) 0117676.7

(51) Int. Cl. A61K 31/70

(52) U.S. Cl. 514/23; 514/53

(57) ABSTRACT

A stable suspension of phosphate in water, which may be used as a basis for dishwashing formulations, comprises a phosphate, such as sodium tripolyphosphate, a carbohydrate, such as sucrose and water in about equal proportions, together optionally with minor proportion, e.g. up to 10% each, of surfactants, stain removers, alkalis and/or other conventional ingredients of automatic dishwashing formulations

The present invention relates to phosphate suspensions which are of particular value in the formulation of liquid dishwash compositions.

Automatic dishwash formulations typically comprise high levels of alkaline sodium tripolyphosphate together with low levels of surfactant, to avoid foaming. This contrasts with manual washing up liquids, which comprise high levels of surfactant and relatively low levels of water soluble builders, and with laundry detergents which contain high levels of surfactant and builder.

Hand washing formulations can readily be obtained as clear stable solutions, usually with the aid of hydrotropes and small amounts of solvent such as ethanol to keep both the surfactant and the builder in solution. This approach has not been adequate to provide the higher levels of builder which are desirable for liquid laundry detergents, which should ideally be about 30% by weight,

The more cost effective builders such as sodium tripolyphosphate are only sparingly soluble in water and neither hydrotropes nor solvents can provide such high levels of builder as solutions. Instead liquid laundry detergents utilise the high levels of surfactant to form a structured surfactant system. The combination of surfactant with a desolubiliser (which may be or comprise the dissolved portion of the builder) forces the former into a lamellar mesophase which has a yield point sufficient to immobilise insoluble particles, maintaining them in suspension when the system is at rest, but low enough to break down when the system is poured or stirred, giving it the bulk properties of a mobile liquid.

Structured surfactants normally require about 10 to 25% surfactant together with an electrolyte as desolubiliser. The desolubiliser can also comprise a carbohydrate (see WO 01/05932).

However the level of surfactant required for automatic dishwash compositions is generally insufficient to provide solid suspending systems.

We have now discovered that water-soluble phosphates and water-soluble carbohydrates form stable solid-suspending systems in water, even in the absence of surfactant. We have further discovered that the suspending systems formed by phosphate and carbohydrate can tolerate the presence of small amounts of surfactant and other ingredients conventionally included in automatic dishwash compositions.

Our invention therefore provides a pourable stable aqueous-based structured system, having solid-suspending properties and comprising as essential structurants a water-soluble phosphate and a water-soluble carbohydrate.

The water-soluble phosphate is preferably a condensed phosphate such as a polyphosphate, e.g. pyrophosphate, tetraphosphate or most preferably tripolyphosphate, or a metaphosphate such as hexametaphosphate, or alternatively may be an orthophosphate. The phosphate is preferably an alkali metal phosphate such as potassium or most preferably sodium, or alternatively may be, for instance, an ammonium phosphate. The preferred phosphate is sodium tripolyphosphate. The preferred carbohydrates are mono and disaccharide sugars such as sucrose, maltose, glucose or fructose. Other sugars which can be used include mannose, ribose, galactose, allose, talose, gulose, idose, arabinose, xylose, lyxose, erythrose, threose, acrose, rhamose and cellobiose. The carbohydrate may be a tri- or tetra-saccharide or a water-soluble polysaccharide such as soluble starch. The term “carbohydrate” as commonly used, and as used herein, includes water soluble non-surfactant derivatives of sugars, and starch, especially redox derivatives such as sugar-derived carboxylic acids and their salts, e.g. gluconic acid, mannic acid, ascorbic acid and alginates or reduced sugars such as sorbitol, mannitol or inositol.

The water, phosphate and carbohydrate are preferably present in approximately equal amounts, e.g. each may conveniently be present in an amount of from 20 to 40% based on the total weight of the composition, preferably 28 to 38%, more preferably 30 to 35%. We particularly prefer that the water, sugar and phosphate should each, independently be present in proportions of 28 to 38%, preferably 30 to 35%, based on the total weight of those three ingredients.

The compositions of our invention may optionally include minor amounts of surfactant e.g. up to 10% by weight of the composition, although higher amounts can be tolerated, and are included provided that the phosphate, carbohydrate and water are present in a sufficient proportion to provide a solid-suspending system in the absence of surfactant. We generally prefer that the amount of surfactant should be less than 8%, more preferably less than 5%, most preferably less than 4%, especially less than 3%, e.g. less than 2%, at least where low foam is a requirement.

The surfactant may be or comprise an anionic surfactant, usually of the type: R(E)ₙAX, where R is a hydrophobic group comprising at least 8 aliphatic carbon atoms, E is an ether group, A is an anionic group, X is a cation and n is 0 or 1. For example the anionic surfactant may comprise an alkyl ether sulphate, alkyl benzene sulphonate, alkyl sulphate, alkane sulphonate, olefin sulphonate, sulphosuccinate, sulphosuccinamate, soap, sarcosinate, tauride, isethionate, alkyl phosphate, alkyl ether phosphate or alkyl ether carboxylate. In each case the hydrophobic group comprises an 8 to 25 carbon alkyl or alkenyl group, or a C₈₋₂₅ alkyl phenyl group or a polypropyleneoxy group. Alkyl or alkenyl groups may be straight or branched chain, primary or secondary and preferably have from 10 to 20 eg. 12 to 14 carbon atoms. Ether groups may comprise glyceryl groups and/or 1 to 20 mol polyoxyethylene groups e.g. 2 to 10 mole. The anionic group is usually a sulphate or sulphonate group, but may also be for example, a phosphate, phosphonate or carboxylate group. The cation is usually sodium but may also be potassium, lithium, annnonium or calcium or other alkali metal or alkaline earth metal or a C₁₋₃ amiine or C₂₋₆ alkanolarline group, such as isopropanolamine or ethanolamine.

The surfactant may optionally be or comprise non-ionic surfactants such as C₂₋₅ alkyl mono or diethenanolamides, amine oxides or surfactants of the type: REOH, where R and E have the same significance as before, for example, 1 to 50 mole ethoxylates such as C₈₋₂₅ alcohol or fatty acid ethoxylates, alkylphenyl ethoxylates, alkyl amine ethoxylates, or glyceryl or sorbitan ester ethoxylates, or preferably polyoxypropylene oxyethylene block copolymers. Ethoxylates typically contain from 2 to 40 eg. 3 to 30 especially 5 to 15 oxyethylene groups. Other non-ionic surfactants include alkyl polyglycosides, sugar esters or amine oxides. The non-ionic surfactants typically have a HLB offrom 5 to 16, e.g. 6 to 15, especially 8 to 14, e.g. 10 to 12. However surfactants with HLB as low as 1 may be used.

The surfactant may be or may comprise an amphoteric or zwitterionic surfactant, usually of the type: RN⁺(R¹)₂(CH₂)ₘA⁻, where R and A have the same significance as before, each R¹ is, independently, hydrogen, a C₁₋₄ alkyl or hydroxyalkyl group or a benzyl group and m is 2 or 3. For example the surfactant may comprise a betaine, amidobetaine, amphoacetate, sulphobetaine or phosphobetaine. Typical examples include fatty alkyl dimethyl betaines and alkyl amidopropyl betaines.

The surfactant may, alternatively be or comprise a cationic surfactant, usually of the type R(E)ₙN⁺(R¹)₃Y⁻, where R, R¹, E and n are as before and Y is an anion. For example the cationic surfactant may be a C₈₋₂₅ straight or branched alkyl or alkenyl or alkylphenyl tri C₁₋₄ alkyl or hydroxyalkyl ammonium salt, or di CI alkyl benzyl ammonium salt, or an C₈₋₂₀, alkyl or alkenyl amido amine. Y may be a halide, e.g. chloride, methosulphate, acetate, citrate, tartrate or other anion capable of forming a water-soluble, non-toxic salt.

Dish washing compositions according to the invention typically comprise low foaming surfactants. They usually contain less than 2% surfactant more usually less than 1% e.g. 0.1 to 0.8% especially 0.2 to 0.7% by weight of the composition.

Compositions may also comprise: stain removers such as phosphonates, enzymes, or bleaches (e.g. chloroisocyanurates); antifoams such as silicone antifoams; fragrances; colourings; alkalis such as sodium carbonate and/or sodium silicate; or other conventional ingredients of automatic dishwash liquids, but preferably not substantial amounts of solvents or hydrotropes.

Alkalis are preferably sufficient to give a pH at 5% dilution greater than 10 e.g. 11 to 12.5. Sodium carbonate is typically present in amounts up to saturation or above, e.g. 0 to 12% especially 0.1 to 10% by weight of the composition. Sodium silicate is preferably present in amounts of at least 3% e.g. 5 to 25% preferably 7 to 15%.

The invention is illustrated by the following examples:

### EXAMPLE I

| Component | Wt % |
|---|---|
| sodium tripolyphosphate | 28 |
| sucrose | 28 |
| Ethylene oxide/propylene oxide block copolymer | 0.4 |
| silicone antifoam | 0.2 |
| sodium chloroisocyanurate | 0.8 |
| fragrance | 0.2 |
| sodium carbonate | 6.4 |
| sodium silicate | 8 |
| water | Balance |

The composition is a readily pourable stable, opaque liquid suspension with good cleaning performance.

### EXAMPLE II

| Component | Wt % |
|---|---|
| Cane sugar | 20 |
| Sodium silicate | 10 |
| Sodium tripolyphosphate | 30 |
| Oxypropylene/oxyethylene block copolymer | 1 |
| “PLURAFAC LF403” | |
| Perfume | 1 |
| Water | Balance |

The above composition was a mobile white opaque liquid having a viscosity of 1.5 Pa s at 21 s⁻¹. It was stable and homogeneous after three months at room temperature.

1) A pourable, stable, aqueous-based, structured system, having solid-suspending properties, and comprising as essential structurants a water-soluble phosphate and a water-soluble carbohydrate.

2) A composition according to claim 1, wherein the water-soluble phosphate is a condensed phosphate.

3) A composition according to claim 2, wherein the phosphate is sodium tripolyphosphate.

4) A composition according to claim 1, wherein the carbohydrate is a mono or disaccharide sugar.

5) A composition according to claim 4, wherein the carbohydrate is sucrose, maltose, glucose or fructose.

6) A composition according to claim 1, wherein the water, phosphate and carbohydrate are each present in an amount of from 20 to 40% based on the total weight of the composition.

7) A composition according to claim 1, wherein the water, sugar and phosphate are each, independently present in proportions of 28 to 38%, preferably 30 to 35% based on the total weight of those three ingredients.

8) A composition according to claim 1, comprising a surfactant in a proportion of up to 10% weight of the composition.

9) A composition according to claim 8, wherein the surfactant comprises an anionic surfactant, of the type: R(E)ₙAX, where R is a hydrophobic group comprising at least 8 aliphatic carbon atoms, E is an ether group, A is an anionic group, X is a cation and n is 0 or 1.

10) A composition according to claim 8, wherein the surfactant comprises a non-ionic surfactant.

11) A composition according to claim 10, wherein the surfactant comprises a surfactant of the type: REOH, where R and E have the same significance as in claim 9.

12) A composition according to claim 11, wherein the surfactant comprises an oxypropylene/oxyethylene block copolymer.

13) A composition according to claim 8, containing 0.1 to 0.8% surfactant by weight of the composition.

14) A composition according to claim 1 and further comprising: stain removers; antifoams; fragrances; colourings; alkalis; and/or other conventional ingredients of automatic dishwash liquids.

15) A composition according to claim 1 further containing claim sodium carbonate in amounts up to 12% by weight of the composition.

16) A composition according to claim 1 further containing sodium silicate in an amount of 7 to 15%, by weight of the composition.
